(19) United States
(12) Patent Application Publication
(10) Pub. No.: US 2024/0144600 A1
(43) Pub. Date: May 2, 2024

(54) CONSTRUCTING TEXTURED 3D MODELS OF DENTAL STRUCTURES

(71) Applicant: INSTITUT STRAUMANN AG, Basel (CH)

(72) Inventors: Alexander SCHMIDT-KRULIG, Chemnitz (DE); Andreas HELBIG, Chemnitz (DE); Julien MARBACH, Montreal (CA); Patrick BERGERON, Montreal (CA)

(21) Appl. No.: 18/389,830

(22) Filed: Dec. 20, 2023

Related U.S. Application Data
(63) Continuation of application No. PCT/IB2022/055804, filed on Jun. 22, 2022.
(60) Provisional application No. 63/213,389, filed on Jun. 22, 2021.

(51) Int. Cl. G06T 17/20; A61C 9/00; G06T 7/00; G06T 11/00; G06T 15/04; G06T 19/20

(52) U.S. Cl. CPC G06T 17/20; A61C 9/0053; G06T 7/0012; G06T 11/001; G06T 15/04; G06T 19/20; G06T 2200/08; G06T 2207/10024; G06T 2207/10028; G06T 2207/30036; G06T 2207/30168; G06T 2210/41; G06T 2219/2004; G06T 2219/2012

(57) ABSTRACT

A method is provided for generating a texture for a three-dimensional (3D) model of an oral structure. The method includes providing the 3D model of the oral structure in the form of a polygon mesh, identifying a set of points located on the polygon mesh, and determining, for each respective point in the set of points, a respective texture value. Each respective texture value is determined by identifying a set of frames, filtering the set of frames to identify a subset of frames, determining a set of candidate texture values for the respective texture value, computing, for each respective candidate texture value in the set of candidate texture values, a quality factor, and computing the respective texture value for the respective point by combining, based on their respective quality factors, candidate texture values selected from the set of candidate texture values.

### CROSS REFERENCE TO RELATED APPLICATIONS

This application is a continuation of International Application No. PCT/IB2022/055804 (WO 2022/269520 A1), filed on Jun. 22, 2022, and claims benefit to U.S. Application No. 63/213,389, filed on Jun. 22, 2021. The aforementioned applications are hereby incorporated by reference herein.

### FIELD

The present disclosure is directed to 3D modeling of dental structures, and in particular, to constructing textured 3D models of dental structures.

### BACKGROUND

The application of digital technologies to dentistry has streamlined treatment and improved patient outcomes in multiple dental specialties. Digital technologies have been successfully employed to supplement or even replace legacy physical and mechanical technologies in oral surgery, prosthodontics, and orthodontics. Such technologies have increased efficiency in traditional methods of care, fostered the development of new treatments and procedures, and facilitated collaboration between practitioners and the dental laboratories that provide the appliances, restorations, and tools utilized in the treatment of patients.

However, without highly accurate digital models of a patient's dentition and soft tissues, the efficacy of dental treatments that involve the application of digital technologies can be compromised. Accordingly, accurate digital models of a patient's oral structures are indispensable to a wide variety of modern dental procedures, and substantial research and development efforts have been directed toward improving dental imaging techniques.

In recent years, intraoral scanners have become many practitioners' preferred tool for imaging a patient's oral tissues and constructing a digital model thereof. Intraoral scanners are imaging devices that can be inserted into a patient's mouth in order to image teeth and soft tissue. An intraoral scanner generates digital impression data by capturing thousands of images of the patient's oral tissues as a practitioner moves the scanner through various locations in the patient's oral cavity. The digital impression data, which consists of the thousands of two-dimensional images and data related to the conditions under which each image was captured, can be processed in order to construct a three-dimensional digital model of the patient's oral structures. As the accuracy of digital models produced by intraoral scanning has improved, the use of intraoral scanners has supplanted alternative techniques for modeling patients' oral cavities, e.g. creating a plaster model and scanning said model with a stationary laboratory scanner.

A number of different imaging techniques are used by intraoral scanners for acquiring a set of images from which a digital model of a patient's oral structures can be constructed. Common techniques employed by intraoral scanners include confocal imaging techniques and stereoscopic imaging techniques. Regardless of the imaging technique employed by the intraoral scanner, software algorithms, e.g. meshing algorithms, are utilized to process the set of images and their corresponding metadata in order to construct the digital model.

The ability to acquire color information data (i.e. data representative of the color of a patient's teeth and gum tissue) during a scan is a significant advantage afforded by the direct imaging of a patient's oral cavity with an intraoral scanner. Scanning a plaster model of the patient's teeth and soft tissue with a stationary laboratory scanner, by contrast, does not allow the acquisition of any data related to the color of a patient's teeth and gum tissue. Patients will assess many dental treatments largely based on the cosmetic outcome, of which color is a substantial component. Accordingly, the incorporation of accurate color information data into a virtual model of a patient's oral structures can facilitate improved treatment outcomes—particularly for restorations involving a veneer, crown, or prosthetic tooth.

Different techniques have previously been used to provide color information data for incorporation into an intraoral scan. One technique involves constructing a triangle mesh that represents the three-dimensional geometry of the patient's oral structures, coloring each vertex of the triangle mesh, and computing a color in the interior of each triangle in the mesh by interpolating the colors of the closest vertices. In theory, such vertex-based triangle mesh coloring techniques can provide adequate results when the triangles of the mesh are small enough to be able to provide a resolution that is high enough to represent the color of the patient's dental structures with sufficient detail. In practice, however, memory and processing power constraints render it impractical or even impossible to generate a triangle mesh that has triangles small enough to be able to provide color information at a sufficiently high resolution when using such triangle mesh coloring techniques.

In particular, such vertex-based triangle mesh coloring techniques have inherent problems associated with computing the correct colors, such as over-blending and wash-out. Furthermore, even if it were possible to overcome such coloring issues, the color sampling resolution of vertex-based triangle mesh coloring is limited to one color per vertex, without possibility of getting color information within the triangles. Such a lack of color sampling resolution can lead to further problems, such as difficulty in identifying important physical features like margin lines (the physical transition between a restoration such as a crown and the natural tooth) and other physical demarcation on the teeth such as the line where the enamel ends and the root begins which is typically along or close where the gumline meets the teeth and which may be visible when the gumline has receded from the teeth). Brute-force techniques to address these problems, e.g. by increasing the resolution of the mesh itself (more vertices and triangles), are not practical and introduce more problems than they would solve. Hardware performance requirements, for example, limit the practicality of such brute-force techniques.

Furthermore, triangle meshes generated from intraoral scan data often include a mix of small and large triangles depending on the geometric details of the modeled object. Triangles are small where the local curvature of the modeled object is high (e.g. at tooth edges) and it is necessary to provide geometric features with high resolution, while triangles are larger where the local curvature is low (e.g. on planar tooth surfaces) and geometric features can be adequately represented using lower resolution (in order to, e.g., save memory and reduce the processing power required to manipulate the triangle mesh). However, the resolution required to adequately represent pictorial features, which are indicated by color information, does not necessarily correspond with the resolution required to adequately represent geometric features, such as tooth curvature. As a result, it can be impossible to adequately represent pictorial features collocated with planar tooth surfaces by simply assigning colors to vertices and/or individual triangles of the triangle mesh. Therefore, while the coloring provided by interpolating a color for each triangle from colors of the closest vertices is very efficient in terms of computation speed, the color accuracy, the color uniformity, and the sharpness of fine details is inadequate.

### SUMMARY

According to an embodiment, a method is provided for generating a texture for a three-dimensional (3D) model of an oral structure. The method includes providing the 3D model of the oral structure, the 3D model of the oral structure being provided in the form of a polygon mesh that includes a number of connected polygons registered in a 3D coordinate system, and identifying a set of points located on the polygon mesh, each respective point in the set of points being defined by a coordinate value in the 3D coordinate system. The method further includes determining, for each respective point in the set of points, a respective texture value. Each respective texture value is determined by identifying a set of frames, filtering the set of frames to identify a subset of frames, and determining a set of candidate texture values for the respective texture value, each candidate texture value corresponding to a respective frame in the subset of frames. Each respective texture value is further determined by computing, for each respective candidate texture value in the set of candidate texture values, a quality factor, and computing the respective texture value for the respective point by combining, based on their respective quality factors, candidate texture values selected from the set of candidate texture values. The method further includes creating a texture atlas, the texture atlas being provided in the form of a two-dimensional (2D) texture image, the 2D texture image including a number of texels, and a mapping between each respective texel in the 2D texture image and a corresponding point located on the polygon mesh in the 3D coordinate system. Each respective texel in the 2D texture image has a value equal to the respective texture value determined for the respective point in the set of points that corresponds to the respective texel.

### BRIEF DESCRIPTION OF THE DRAWINGS

Subject matter of the present disclosure will be described in greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 illustrates a 2D to 3D mapping process;

FIG. 2 illustrates an intraoral scanner designed to acquire scan data for constructing a 3D virtual model of dentition and oral tissues;

FIG. 3 illustrates an intraoral scanner hardware platform including the intraoral scanner of FIG. 2;

FIG. 4 illustrates an alternative intraoral scanner hardware platform including the intraoral scanner of FIG. 2;

FIG. 5 is a flow diagram illustrating a process for constructing a colored 3D virtual model of dentition and oral structures;

FIG. 6 is a flow diagram illustrating a 3D scan process according to an embodiment;

FIG. 7A is a flow diagram illustrating an image capture process according to an embodiment;

FIGS. 7B and 7C illustrate the impact of scanner movement on the capture position of consecutive images of a single frame and the impact of a channel shift correction;

FIG. 8 illustrates a process for constructing a texture atlas and a mapping between a texture atlas and a 3D mesh;

FIG. 9 illustrates a process for creating frame objects;

FIG. 10A illustrates a process for computing color for each point in a 3D mesh, i.e. for computing a color for each texel of a texture atlas;

FIG. 10B illustrates a single tile including two triangles of a triangle mesh and 16 different texels of a texture atlas that maps to the triangle mesh;

FIG. 10C illustrates a texture atlas at different levels of resolution;

FIG. 11A illustrates a textured, i.e. colored, 3D virtual model of a tooth;

FIG. 11B illustrates a 3D triangle mesh that corresponds to the textured 3D virtual model of the tooth of FIG. 11A;

FIG. 11C illustrates a 3D point cloud model that represents the tooth modeled in FIGS. 11A and 11B;

FIG. 12 is a block diagram of an exemplary processing system, which can be configured to perform operations disclosed herein;

FIG. 13A illustrates a camera perpendicularity test for use in computing color for a point in a 3D mesh;

FIG. 13B illustrates a distance test for use in computing color for a point in a 3D mesh;

FIG. 13C illustrates a test, for use in computing color for a point in a 3D mesh, to determine whether a point of interest lies within a view frustum of an image; and

FIG. 13D illustrates an occlusion test for use in computing color for a point in a 3D mesh.

### DETAILED DESCRIPTION

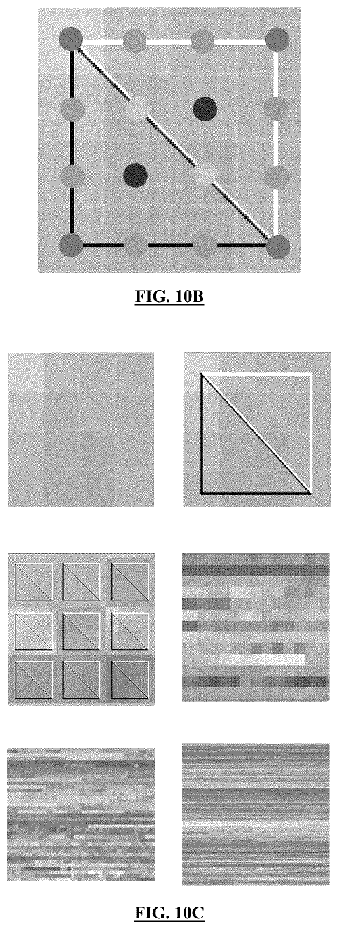

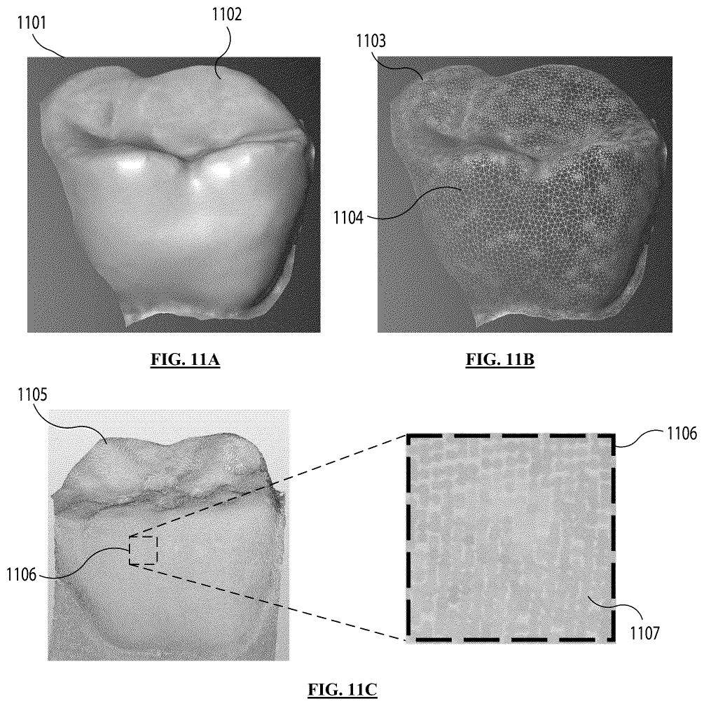

The present disclosure relates to 3D modeling of dental structures, e.g. performed by intraoral scanners designed to provide a digital representation of a patient's teeth, and in particular, to constructing color information in a 3D model of dental structures. In an embodiment, a 3D model comprises a point cloud (such as shown in FIG. 11C at 1105), and such color information may take the form of computed color values for the points 1107 of the point cloud. In an alternative embodiment, the 3D model comprises a 3D polygon mesh (such as shown in FIG. 11B at 1103), and the color information may take the form of computed color values for the vertices of the polygon mesh, or alternatively may take the form of computed colored texture tiles to be applied to the polygon mesh. In particular, the method contemplates generating a 3D model of the dental structures and subsequently computing the color information and applying the color information to the 3D model. As a result of the techniques described herein, an intraoral scanner and its companion software can generate, on a computer screen, a full-color, 3D virtual model of a patient's dentition and soft tissue. In particular, the techniques described herein improve the on-screen realism of 3D virtual models by providing for a photo-realistic rendering of a 3D model generated by an intraoral scan.

Due to the cosmetic nature of many dental treatments, such as restorations involving a veneer, crown, or prosthetic tooth, and the substantial importance of color to the cosmetic outcome of such treatments, highly detailed color data adds significant value to 3D models. For example, highly detailed color information in a 3D digital model of a patient's dental structures can enable a dental laboratory to better match the color of artificial teeth and/or gum tissue in a prosthetic component to be installed in the patient's mouth. Furthermore, highly detailed color information can also assist a practitioner in ascertaining the precise location of a boundary between a patient's teeth and soft tissue in a 3D digital model. As a result, highly detailed color information can facilitate the clear identification of clinically relevant details, thereby improving diagnostic accuracy and the overall quality of digitally designed dental restorations.

Techniques described herein provide improved colored 3D models that are more realistic than colored 3D models constructed by traditional methods. The improved colored 3D models facilitate improved treatment outcomes by, e.g., providing dental laboratories with color details critical for proper design of dental restorations and prostheses, and enable both patients and practitioners to better visualize existing dental pathologies and thereby make more informed decisions regarding potential treatments. In addition, techniques described herein include algorithms that provide for the computation of a color or texture to be mapped to a virtual 3D model of a patient's oral structures with reduced processing power and memory storage requirements.

In contrast to the above mentioned vertex coloring, solutions described herein construct color information for 3D models based on a combination of color value(s) obtained from a set of frame images selected from actual scan images captured of the actual object (such as oral structure, e.g., a dentition, a tooth or teeth, gum tissue) modeled by the 3D model. Each selected frame includes individual color channel data (e.g., R, G, and B) and depth information (such as a UV channel) contained in a single 3D composite image. Preferably, each selected frame is selected based on a computed quality factor. According to one aspect, for each of a plurality of points in the 3D model of the object, the color information from each corresponding point of the scanned object in the plurality of selected frames is combined to calculate the color information value for the respective point (e.g., a point in a 3D point cloud model (see FIG. 11C), or a vertex in a 3D mesh model (see FIG. 11B)). FIG. 11C shows a colored point cloud 1107 with individual colored points 1109 (as best seen in magnified section 1108) that are colored. FIG. 11B shows a triangle mesh 1103 comprising a set of triangles 1106 formed by connecting points (called vertexes 1105).

According to another aspect, for a 3D mesh model, in addition to the ability to color the vertices of the polygons of the mesh, a digital texture, independent of the polygon mesh and containing color information, is constructed, which is then mapped to the polygon mesh. FIG. 11A shows a colored texture 1102 rendering of the scanned object. As a result, the resolution of the color information is not tethered to the resolution of the geometric information, and very fine color details can be provided even for surfaces consisting of very coarse geometric details (e.g. large triangles or other polygon shapes). Accordingly, the difficulty in identifying important physical features, like margin lines, as a result of the lack of the color sampling resolution can be addressed via the texturing techniques described herein.

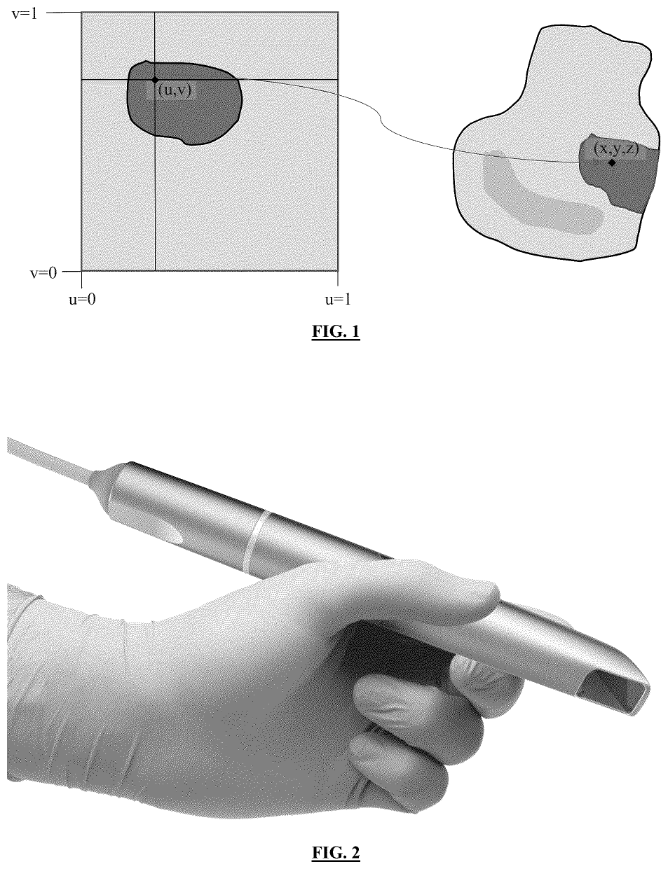

Texture mapping, i.e., the approach for applying texture to the 3D model consists of mapping an image or collection of images, i.e. a texture image, onto the 3D model. To map pixels of the texture image onto the 3D model, a pixel of the texture image (i.e. a “texel”) is identified for each visible pixel of the 3D model and then mapped to that point. This computation is made when rendering the 3D scene on the screen and is facilitated by a precomputed mapping that, for each point in the 3D model (e.g., each vertex in a 3D mesh model) with 3D coordinates (x,y,z), gives the 2D coordinates (u,v) of its matching pixel in the texture image. This mapping between a 2D image and a 3D image is illustrated in FIG. 1.

The state of the art when generating a digital texture image is to partition the 3D model into multiple patches, which are often relatively large in terms of surface area, and find, in a set of color images captured during the scan of the object, the best matching image covering each patch. Once the best image is found for a patch, the image is projected onto the patch and the 2D (u,v) coordinates of the vertices of the patch are inferred. The image is then cropped to keep only the region of the image that maps to the patch of the 3D model. Once this process is completed for all patches of the model, the final texture image is produced by assembling side by side all the cropped sub-regions of projected images. However, applying such techniques to virtual 3D models created with intraoral scanners can be problematic. In particular, discrepancies in color and illumination are often visible from one patch to another due to the discrepancies in color and illumination present from one cropped image used to provide texture for one patch to another cropped image used to provide texture for a different patch. Furthermore, such techniques are prone to introducing baked-in specular effects (e.g., caused by reflections present in an image used to provide texture for a first patch and in an image used to provide texture for an adjacent patch) and visible stitchings (e.g., at boundaries of different patches).

In contrast to traditional methods of constructing colored or textured 3D models of dental structures, solutions described herein operate differently. In particular, solutions described herein generate a point color or texture whose individual pixels specify a color for each respective point of a 3D scan, the color of each specified pixel being computed by sampling colors from a set of the N best 2D images for each respective point. As a result of the techniques employed by the solutions described herein, discrepancies in color and illumination from one portion of the textured 3D model to another portion of the textured 3D model are reduced, baked-in specular effects are reduced or even altogether eliminated, and visible stitchings can be avoided.

The techniques disclosed herein involve performing a 3D scan during which both depth and color data, which is used to construct a 3D model, is acquired. During the 3D scan, one or more cameras, for example in an intraoral scanner, records 2D images and associated metadata. An image, in the context of this discussion of image scan capture, is any 2D image captured by a camera, which could be an individual R, G, B or UV image (discussed hereinafter), which could be combined into a container called a “composite image” in which stores in a single object or file the R, G, B and UV image information captures at a given scanner location by a given camera. In a composite image, each color channel is a “layer” of the composite image. The scanner always knows its 3D position and orientation in 3D space. Every few millimeters, the scanner will record a frame. A frame comprises metadata and a set of composite images captured at a given scanner location. In a system that includes a single camera, a frame includes metadata and a single composite image. In a system that includes multiple cameras in the scanner that simultaneously capture the same scene from different view points, a frame comprises metadata and a composite image captured from each camera when the camera is at the given location defined by the scanner position, orientation, etc. A frame contains metadata that includes, e.g., a timestamp, the scanner's position (e.g. measured from an origin point of the scanner at the initiation of the scan), the scanner's orientation, and the scanner movement speed (i.e. the speed of movement of the scanner during the recording of the frame). As just mentioned, the frame may include a number of images, e.g. 1 composite image for each of the scanner's cameras. As an example, a scanner may include one camera for acquiring depth data (a camera designed to acquire an ultraviolet image) and three cameras for acquiring color data (cameras designed to acquire red, green, and blue images). Alternatively, a frame may include multiple images acquired by the same camera, e.g. with different illumination conditions. The frames are then stored in memory. For example, the metadata can be written in a binary file, and all of the images can be saved separately, e.g. as JPGs (to save disk space). To compute a 3D model (such as point cloud or textured mesh), all frames and associated images must be loaded into memory.

The metadata can include, e.g., an image capture location (which is defined in a global 3D coordinate system), a scanner movement speed (i.e. a speed with which the intraoral scanner is moving during the capture of a particular image or frame consisting of multiple images), an orientation of the intraoral scanner during the capture of a particular image. The 2D images can include both depth images (e.g. images that record data used to determine the 3D structure of the object that is being scanned/imaged) and color images (e.g. images that record data pertaining to the color of the object that is being scanned/imaged). In some techniques, the same images can serve as both depth images and color images, while in other techniques the images that are used to construct the 3D structure of the virtual model and the images that are used to compute the color of the virtual model are entirely separate.

Following and/or during the scan, images that include depth data are provided to an algorithm that computes a 3D geometry of the object being scanned/imaged. The algorithm determines, based on the multitude of images, a point cloud in the global coordinate system (i.e. the same global coordinate system in which the position of the scanner during the scan is defined). The point cloud represents the 3D geometric structure of the scanned/imaged object. In an embodiment, coloring can occur directly on the point cloud using the processes described hereinafter.

Alternatively, following the construction of the point cloud, a meshing algorithm computes a 3D mesh based on the point cloud. The 3D mesh adds topology to the 3D geometry represented by the point cloud. In an embodiment, once the 3D mesh has been constructed, the vertices of the polygons of the mesh can be colored according to the processes described hereinafter.

Alternatively, or in addition, a texturing algorithm may be utilized to determine a texture atlas (i.e. a texture image containing color information, which includes a multitude of texels (each “texel” being a pixel of the texture image), and a mapping between the texture atlas and the 3D mesh). Unlike the coloring of points in a point cloud 3D model, or coloring of vertices of the polygon mesh, the texture atlas solution allows individual texels within the texture atlas to be colored as well, such then when rendered over the corresponding 3D mesh, individual pixels within the polygons may be individually colored to allow for a more accurate and authentic colorization. Solutions described herein identify, for each point on the 3D mesh (which may include points within the mesh polygons) that corresponds to a texel in the texture atlas, a color. In such embodiment, in order to display/render the textured 3D model on a display, the texture atlas, the 3D mesh, and the mapping therebetween can be provided to a rendering engine.

The colorization techniques for each of the above-described 3D coloring models —that is, coloring the points in a point cloud 3D model, coloring the vertices in a 3D mesh model, and coloring the points (texels) of a texture atlas to be applied to a 3D mesh model —involve a core process of calculating the color for each of the points/texels to be colored. In order to compute the color of a single respective 3D point, the set of images obtained from a scan capture of the actual object being modeled (e.g., the frame images from the scan, or composite image or other processed image(s) generated therefrom) that include the point are identified by determining whether the point lies in the view frustum of each of the images. In addition, for each of the identified images for which the point lies in the view frustum thereof, an occlusion test is performed to determine whether, in each respective image, the point is occluded by some other structure (e.g. another point or by some other polygon (e.g., triangle) in the polygon mesh). Then, for each image that is determined to include an unobstructed view of the point (i.e. for each image having a pixel that corresponds to the point), a quality factor for the color of the point in the image is computed, e.g. based on camera perpendicularity, scanner movement speed (i.e. a measured speed of movement of the scanner during acquisition of the frame), focal distance, and other criteria. The N best (e.g. 15 best) images (as determined according to the weighted quality factors) are kept and the rest are discarded. The final color of the point is then determined as the weighted average of the N-best images.

In various different embodiments, methods and systems described herein can perform different tests and/or calculations—which serve to determine the suitability of different images for computing the color of a 3D point—in different sequences. For example, in some embodiments, an occlusion test can be performed prior to considering the view frustum. Additionally, other considerations can be taken into account before either or both of the occlusion test and view frustum determination. For example, all images with a focal distance or scanner movement speed that exceeds a corresponding threshold value could be excluded prior to performing the view frustum analysis and/or the occlusion test. Various other sequences not specifically identified here could also be employed in different embodiments.

In certain embodiments, the selection of the N best 2D color images can be performed in a 2-stage filtering process. For example, in order to identify the N best 2D color images, a coloring algorithm can first perform a coarse filtering to eliminate all 2D color images that do not include the point in the 3D mesh or in which the point in the 3D mesh is obscured. In order to rapidly eliminate 2D color images in which the point on the 3D mesh is occluded/obstructed, an Octree can be used. The coarse filtering can thereby eliminate images based on hard criteria, i.e. criteria that determine whether or not an image includes any color data that corresponds to a particular point in the 3D mesh. The coarse filtering can also eliminate 2D color images that were captured from a location that is greater than a threshold distance from the point in the 3D mesh. The coarse filtering can also eliminate other 2D images based on other criteria, e.g. soft criteria (i.e. criteria used to assess the suitability of color data that corresponds to a particular point).

Following the coarse filtering of the 2D color images, the remaining 2D color images—all of which have a view frustum that includes the point in the 3D mesh, can be further filtered, via a fine filtering process, to identify the N best images to use for the respective point in the 3D mesh. The fine filtering process includes assigning a suitability score to each of the remaining 2D color images in order to determine their suitability for use in contributing to the color to be provided for the respective point in the 3D model or texture atlas. Once suitability scores are assigned, the best N images (e.g. all images having an above-threshold suitability score or the N highest suitability score images) are identified, and the colors of those images are weighted in order to determine a color of a point in the 3D model or a texel in the texture atlas that is mapped to the respective point in the 3D space. In scoring the remaining images, positions of different cameras that acquired images for different color parameters (e.g. RGB) and or positions of cameras at different points in time at which data corresponding to different color parameters was acquired can be computed, e.g. from an assumed position at which depth data was actually acquired by using scanner movement speed, scanner position, and scanner orientation from metadata. The fine filtering can thereby select, from a set of images that include color data that corresponds to the particular point in the 3D mesh, the best images for coloring the point based on soft criteria.

Techniques according to an aspect of the present disclosure compute point colors in a 3D point cloud model of an object such as an oral structure. In this technique, a process identifies a set of points in the plurality of points of the 3D model, each respective identified point in the 3D model being defined by a coordinate value in the 3D coordinate system. The process determines, for each identified point in the 3D model, a respective color information value. According to the technique, the respective color information value is determined by identifying a set of images captured from an image scan of at least a portion of the oral structure, the identified set of images each comprising a corresponding point that corresponds to the respective point in the 3D model and each having associated color information, combining the color information associated with the corresponding point in each of the identified scan images into a color information value, and associating the combined color information value with the respective color information value of the respective point in the 3D model.

Techniques according to another aspect of the present disclosure compute vertex colors of vertexes of polygons of a 3D polygon mesh. In this technique, a process identifies a set of vertexes in a 3D polygon mesh model. The process determines, for each identified vertex in the 3D model, a respective color information value. According to the technique, the respective color information value is determined by identifying a set of images captured from an image scan of at least a portion of the oral structure, the identified set of images each comprising a corresponding point that corresponds to the respective vertex in the 3D model and each having associated color information, combining the color information associated with the corresponding point in each of the identified scan images into a color information value, and associating the combined color information value with the respective color information value of the respective point in the 3D model.

Techniques according to another aspect of the present disclosure compute a color for various 3D points of a 3D mesh model, via use of a texture atlas, but each respective point for which a color is computed does not have to lay on the geometry of vertices itself. Instead, the various 3D points for which color is computed can be located on the edges or the interior of the surface primitives that make up the 3D mesh model, e.g. on the edges or the interior of the triangles of a 3D triangle mesh. The techniques according to the present disclosure can also compute color for areas where the scan did not create 3D geometry. For example, the techniques according to the present disclosure can compute colors for flat areas inside the polygons of a polygon mesh, thereby allowing a high resolution texture to be provided even for a 3D mesh with very large polygons (such as triangles). As a result, the approach of the present techniques is independent of the topology resolution, which allows the color data generated thereby to survive position smoothing (i.e. moving the points a bit in space) and topology edits—such as removal of points and polygons. The present techniques can also be used to color regions that were created after the scan in order to, for example, fill holes.

According to embodiments where the 3D model is a 3D triangle mesh textured using a texture atlas, an explicit point/normal exists at each vertex of each triangle, an infinity of implicit points/normal exist along each edge of each triangle, and an infinity of implicit points/normal exist inside each of the triangles. A texture resolution for the mesh can be determined that corresponds to a density of the scanned images, i.e. a number of texels can be specified for each triangle. For example, ten texels—one for each vertex, two for each edge, and one for the interior region—can be specified for a given triangle. Two triangles can be combined to form a texture tile that includes 4×4 (i.e. 16) texels (i.e. 2 independent vertices, 2 shared vertices, 2 points for each of the five edges of the two triangles, and 1 point for each interior region of the two triangles). Thereafter, 2D coordinates in a 2-dimensional (2D) image coordinate system can be assigned to each triangle so that they correctly map to the appropriate texture tile.

In constructing texture tiles of a texture atlas, triangles are forced to align together. In particular, two triangles in a pair are aligned together by rotating vertex/normal/color indices such that the shared edge is always the triangle's first edge. By assuming that the shared diagonal is always the first edge of both triangles, the remainder of the algorithm for assembling the tile does not contend with any edge case, does not branch, and is straightforward to follow. A disadvantage of rotating indices within the triangles is that it modifies the topology of the input mesh. However, such disadvantage is not particularly problematic in the generation of the texture atlas for a 3D mesh in the present context.

Inefficiencies in the texture atlas, in terms of wasted texels, can be reduced using certain optimization techniques. A first optimization technique is to assemble a large number of individual triangles of the triangle mesh into tile strips as opposed to simply pairing triangles for a tile. When two adjacent triangles are combined to form a single tile, the texels along the shared edge of the triangles (i.e. the texels along the diagonal of the tile) are shared but the texels for the other two edges of each triangle are not shared, in the texture atlas, with the other triangles with which said edges are shared in the mesh. Accordingly, the texture map includes a number of duplicate texels, i.e. texels from different texture tiles that correspond to the same point in the 3D mesh. If correct tiles are placed next to one another (such that the texels on the edge of each tile correspond to the same edge and vertices in the 3D mesh) to form a tile strip, duplicate texels can be removed, and (u,v)coordinates in the texture atlas can be shared along 2 out of 3 triangle edges. To generate the texture atlas in this fashion, instead of laying unrelated tiles next to each other in the horizontal direction, tiles that share the same set of texels along an edge of the tile are laid out adjacent to one another in the horizontal direction such that the shared set of texels are overlapped in the atlas. Adjacent tiles can be laid out next to one another in this manner until a border of the texture is reached (and a new strip can then be begun at an opposite border of the texture). Such technique can reduce the size of the texture file by about 25%—a non-negligible amount of memory.

A second optimization technique when generating a texture atlas is to reduce tile sizes based on color uniformity. At the expense of color quality loss, the tile contents can be inspected for color uniformity, and the tiles can be compressed, i.e. the texel density can be reduced, if the color uniformity is above a threshold. For example, a 4×4 texel tile can be compressed to a 2×2 texel tile (if colors are roughly uniform), or even compressed to a single texel if the colors are very uniform within the tile.

According to an aspect of the disclosure, a method is provided for generating a texture for a three-dimensional (3D) model of an object such as an oral structure. The method includes providing the 3D model of the object such as the oral structure, the 3D model of the object being provided in the form of a polygon mesh that includes a number of connected polygons registered in a 3D coordinate system, and identifying a set of points located on the polygon mesh, each respective point in the set of points being defined by a coordinate value in the 3D coordinate system. The method further includes determining, for each respective point in the set of points, a respective texture value. Each respective texture value is determined by identifying a set of frames, filtering the set of frames to identify a subset of frames, and determining a set of candidate texture values for the respective texture value, each candidate texture value corresponding to a respective frame in the subset of frames. Each respective texture value is further determined by computing, for each respective candidate texture value in the set of candidate texture values, a quality factor, and computing the respective texture value for the respective point by combining, based on their respective quality factors, candidate texture values selected from the set of candidate texture values. The method further includes creating a texture atlas, the texture atlas being provided in the form of a two-dimensional (2D) texture image, the 2D texture image including a number of texels, and a mapping between each respective texel in the 2D texture image and a corresponding point located on the polygon mesh in the 3D coordinate system. Each respective texel in the 2D texture image has a value equal to the respective texture value determined for the respective point in the set of points that corresponds to the respective texel.

In the method for generating the texture for the 3D model of the oral structure, the set of points located on the polygon mesh can include, for each respective polygon in the polygon mesh, at least one point. The set of points located on the polygon mesh can include, for each respective polygon in the polygon mesh, at least one vertex point, at least one edge point, and at least one interior point. In some embodiments, each polygon in the polygon mesh is a triangle, and wherein the set of points located on the polygon mesh includes, for each respective triangle in the polygon mesh, three vertex points, at least three edge points, and at least one interior point.

In the method, each frame in the set of frames can include a depth image and a composite color image, and the 3D mesh is a 3D mesh constructed using depth data from the respective depth images. The composite color image can include a plurality of color channels.

In the method, determining each respective candidate texture value that corresponds to a respective frame in the subset of frames can include determining, for each respective color channel of the plurality of color channels of the composite color image of the respective frame, a color channel contribution, and combining each respective color channel contribution to provide the respective candidate texture value. The composite color image of each frame in the subset of frames can be a combination of monochrome images, each monochrome image corresponding to a respective color channel of the plurality of color channels. Determining the color channel contribution for each respective color channel of the composite color image can include determining, based on a camera position in the 3D coordinate system that corresponds to the monochrome image corresponding to the respective color channel and the coordinate value in the 3D coordinate system of the respective point for which the respective texture value is computed, a pixel in the monochrome image and providing a pixel value of the determined pixel as the color channel contribution for the respective color channel. Each respective monochrome image of each composite image can be independently associated with a respective camera position in the 3D coordinate system.

In the method, filtering the set of frames to identify the subset of frames can include performing, for each respective frame in the set of frames, at least one of: a camera perpendicularity test that analyzes a degree of perpendicularity between a camera sensor plane corresponding to the respective frame and a normal of the respective point located on the polygon mesh, a camera distance test that analyzes a distance, in the 3D coordinate system, between a camera capture position corresponding to the respective frame and the respective point located on the polygon mesh, a view frustum test that determines whether the respective point located on the polygon mesh is located in a view frustum corresponding to the respective frame, or an occlusion test that analyzes whether the point located on the polygon mesh is, in an image corresponding to the respective frame, obstructed by other surfaces of the polygon mesh.

In the method, computing, for each respective candidate texture value in the set of candidate texture values, a quality factor can include assigning, for each respective frame in the set of subframes, weighting factors based on at least one of: a degree of perpendicularity between a camera sensor plane corresponding to the respective frame and a normal of the respective point located on the polygon mesh, a distance, in the 3D coordinate system, between a camera capture position corresponding to the respective frame and the respective point located on the polygon mesh, a scanner movement speed corresponding to the respective frame, or a degree of whiteness of the respective candidate texture value.

In the method, computing the respective texture value for the respective point by combining, based on their respective quality factors, candidate texture values selected from the set of candidate texture values can include selecting a subset of the candidate texture values based on their respective quality factors and averaging individual color channel values provided by each candidate texture value in the subset of candidate texture values.

According to an aspect of the disclosure, a non-transitory computer readable medium is provided having processor-executable instructions stored thereon. The processor-executable instructions are configured to cause a processor to carry out a method for generating a texture for a three-dimensional (3D) model of an object such as an oral structure. The method includes providing the 3D model of the object, the 3D model of the object being provided in the form of a polygon mesh that includes a number of connected polygons registered in a 3D coordinate system, and identifying a set of points located on the polygon mesh, each respective point in the set of points being defined by a coordinate value in the 3D coordinate system. The method further includes determining, for each respective point in the set of points, a respective texture value. Each respective texture value is determined by identifying a set of frames, filtering the set of frames to identify a subset of frames, and determining a set of candidate texture values for the respective texture value, each candidate texture value corresponding to a respective frame in the subset of frames. Each respective texture value is further determined by computing, for each respective candidate texture value in the set of candidate texture values, a quality factor, and computing the respective texture value for the respective point by combining, based on their respective quality factors, candidate texture values selected from the set of candidate texture values. The method further includes creating a texture atlas, the texture atlas being provided in the form of a two-dimensional (2D) texture image, the 2D texture image including a number of texels, and a mapping between each respective texel in the 2D texture image and a corresponding point located on the polygon mesh in the 3D coordinate system. Each respective texel in the 2D texture image has a value equal to the respective texture value determined for the respective point in the set of points that corresponds to the respective texel.

According to an aspect of the disclosure, a system is provided for generating a texture for a three-dimensional (3D) model of an object such as an oral structure. The system includes processing circuitry configured to provide the 3D model of the object, the 3D model of the object being provided in the form of a polygon mesh that includes a number of connected polygons registered in a 3D coordinate system. The processing circuitry is further configured to identify a set of points located on the polygon mesh, each respective point in the set of points being defined by a coordinate value in the 3D coordinate system, and determine, for each respective point in the set of points, a respective texture value. Each respective texture value is determined by identifying a set of frames, filtering the set of frames to identify a subset of frames, and determining a set of candidate texture values for the respective texture value, each candidate texture value corresponding to a respective frame in the subset of frames. Each respective texture value is further determined by computing, for each respective candidate texture value in the set of candidate texture values, a quality factor, and computing the respective texture value for the respective point by combining, based on their respective quality factors, candidate texture values selected from the set of candidate texture values. The processing circuitry is further configured to create a texture atlas, the texture atlas being provided in the form of a two-dimensional (2D) texture image, the 2D texture image including a number of texels, and a mapping between each respective texel in the 2D texture image and a corresponding point located on the polygon mesh in the 3D coordinate system. Each respective texel in the 2D texture image has a value equal to the respective texture value determined for the respective point in the set of points that corresponds to the respective texel.

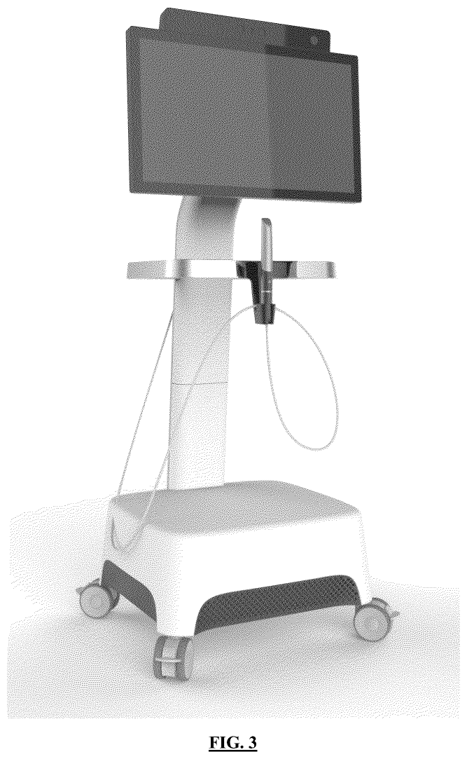

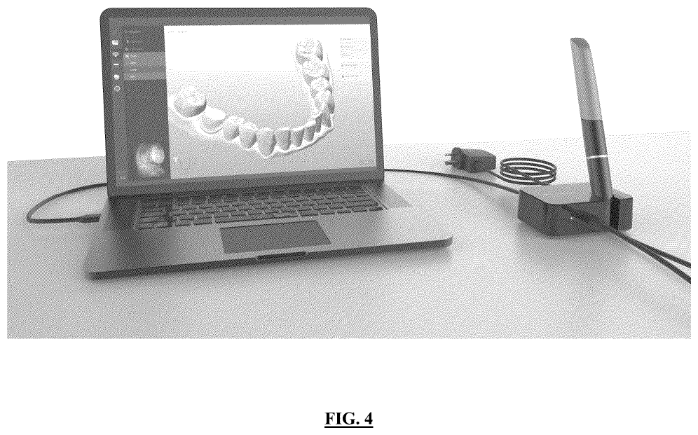

FIG. 2 illustrates an intraoral scanner designed to acquire scan data for constructing a 3D virtual model of dentition and oral tissues. The intraoral scanner includes a handpiece in which multiple cameras and an illuminating light source are disposed. The cameras can include, e.g., a camera configured to acquire images in which ultraviolet light is projected and red, green, and blue monochrome cameras (configured to capture red, green, and blue monochrome images). The illuminating light source can be configured to project ultraviolet pattern light as well as white or red, green, and blue (RGB) light. The UV light and the white/RGB light can be provided from different light sources. The intraoral scanner additionally includes a number of different sensors that are configured to capture data, as well as processing circuitry configured to associate data captured by the sensors with images captured by the cameras, e.g. by associating both the data and the images with a timestamp. The sensors include position, orientation, and speed sensors, which can themselves include one or more accelerometers and/or one or more gyroscopes. FIG. 3 illustrates an intraoral scanner hardware platform including the intraoral scanner of FIG. 2. The hardware platform of FIG. 3 additionally includes a cart and a display mounted on the cart. The hardware platform of FIG. 3 can also include additional processing circuitry configured to process data acquired by the intraoral scanner of FIG. 2, e.g. processing system such as that described in FIG. 12. FIG. 4 illustrates an alternative intraoral scanner hardware platform including the intraoral scanner of FIG. 2. The alternative hardware platform of FIG. 4 includes a laptop computer to which the intraoral scanner is connected. The laptop computer can include additional processing circuitry configured to process data acquired by the intraoral scanner of FIG. 2, e.g. processing system such as that described in FIG. 12. As an alternative to including additional processing circuitry configured to process data acquired by the intraoral scanner, both the hardware platform of FIG. 3 and the alternative hardware platform of FIG. 4 can be connected, via a data connection, to such additional processing circuitry, e.g. located in the cloud. Alternative platforms may include any combination of hardware, computational hardware and software designed to perform the scan image capture and image processing described herein.

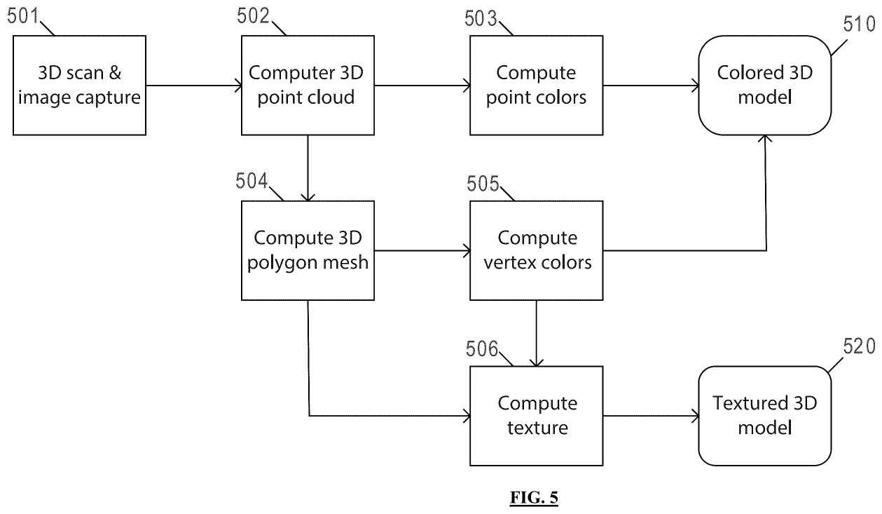

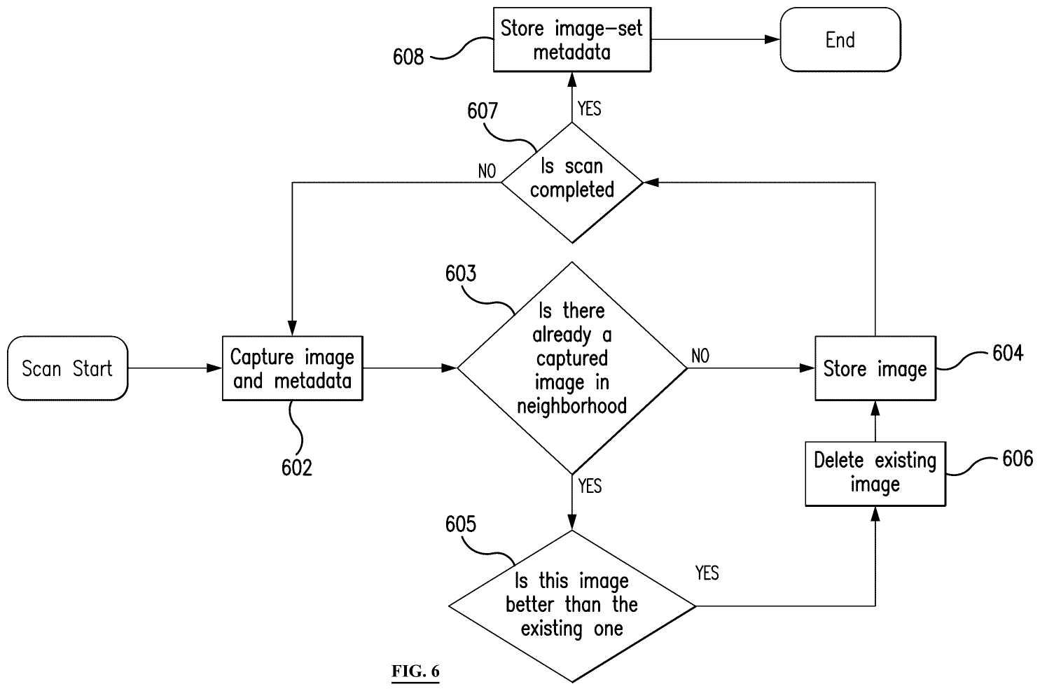

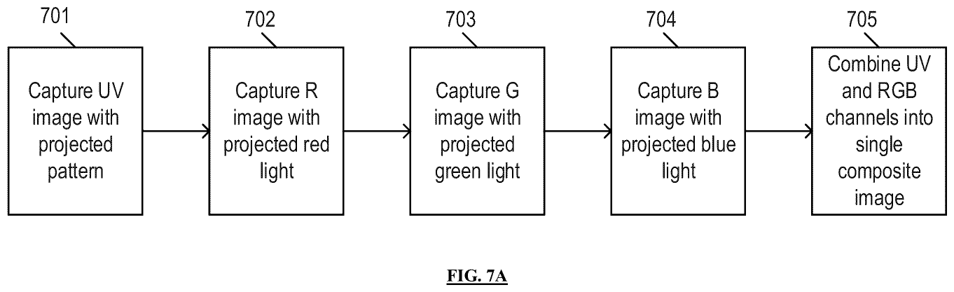

FIG. 5 is a flow diagram illustrating a process for constructing a textured 3D virtual model of one or more object(s) or area of interest (hereinafter, “object”), such as the dentition and oral structures of a dental patient. At 501, a 3D scan is performed, e.g. by the intraoral scanner of FIG. 2. The 3D scan process includes repeatedly capturing images in a sequence, each sequence of images constituting a frame. An example 3D scan process and an example image capture process are illustrated and described in more detail in FIGS. 6 and 7, respectively, and their corresponding descriptions provided herein below.

At 502, a 3D point cloud is computed based on the 3D scan and image capture performed at 501. In an embodiment, the intended format of the 3D scan model is a point cloud. In such embodiment, at 503, color information for the points in the point cloud is calculated and applied to construct a colored 3D model 510 in the form of a colored point cloud.

In an alternative embodiment, the 3D model may be a 3D mesh comprising a plurality of polygons such as triangles or other shapes. In such embodiment, each polygon in the 3D mesh is defined by a set of points (each such point called a “vertex” when referring to the point in the 3D mesh) and a set of edges connecting the set of points around the perimeter of the polygon. In a polygon mesh, each polygon comprises at least 3 edges, and many of the polygons are positioned in the mesh so as to share two vertices and an edge with an adjacent polygon. For illustrative purposes, the discussion shall be presented in terms of a triangle mesh—that is, a 3D triangle mesh that is constructed by a plurality triangles connecting points in the point cloud 3D scan model, where the points are the triangle vertices. It is to be understood that the mesh may be constructed using other polygon shapes, such as quadrilaterals (4 vertices, 4 edges). In a triangle mesh embodiment, at 504, a 3D triangle mesh is computed based on the 3D scan and image capture performed at 501 and point cloud computed at 502. The 3D triangle mesh is computed by a meshing algorithm. The meshing algorithm receives the point cloud as an input and computes a triangle mesh therefrom. As an alternative to triangle meshes, the process can also use different meshing algorithms whereby the point cloud is transformed into a mesh constructed from alternative polygon primitives. The process can also use an algorithm that transforms the point cloud into a 3D mesh constructed from other surface primitives, e.g. parametric surfaces. At 505, the color for each vertex in the 3D triangle mesh is computed. Following the computation of the points for the point cloud at 503 or for the vertices for the 3D triangle mesh at 505, a colored 3D model (point cloud or mesh) is generated at 510.

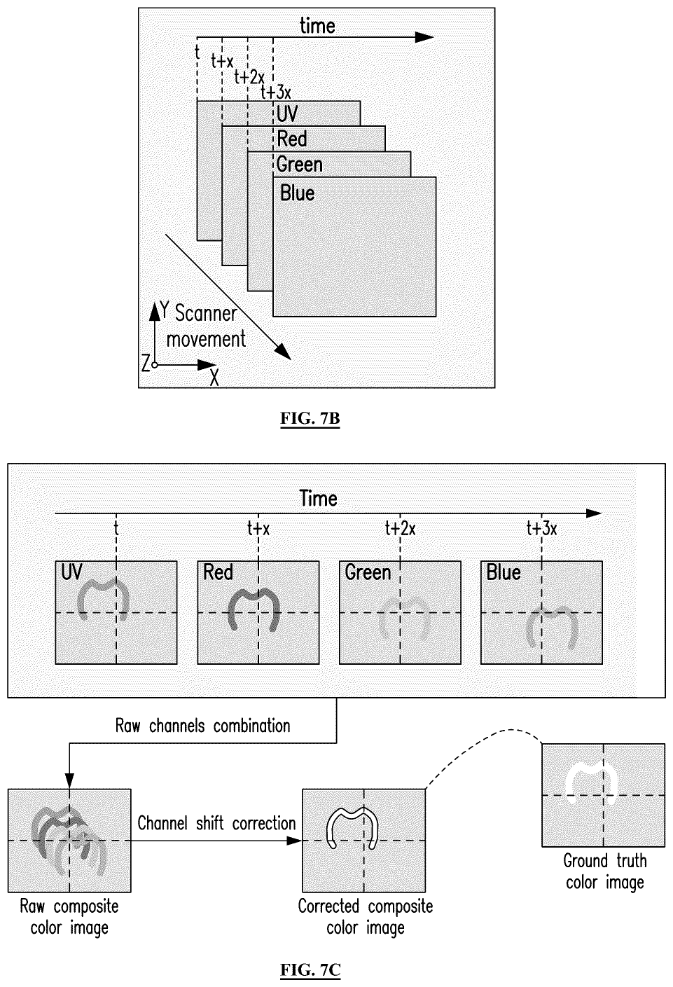

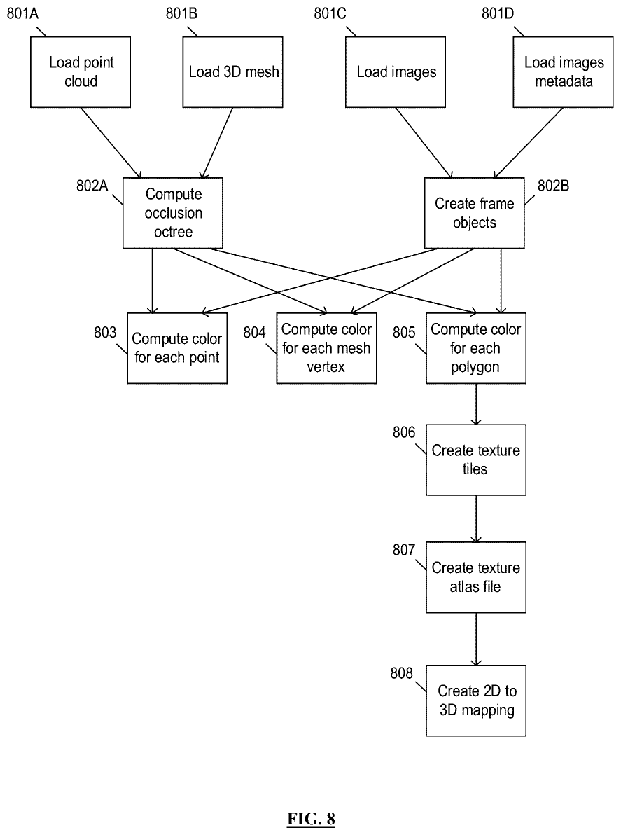

In an alternative embodiment, the 3D model may be a 3D mesh with a texture that maps to the 3D mesh according to a texture atlas, whereby the texture atlas includes texture points that map to points on the edges and/or within the polygons of the 3D mesh rather than only to the vertices of the mesh. Instead of, or in addition to, calculating the color information for the polygon vertices, at 505, texture is computed for the 3D mesh. The texture computation process is illustrated and described in more detail in FIGS. 8-11 and their corresponding descriptions provided herein below. Following the computation of the texture for the 3D mesh, a textured 3D mesh is rendered and output at 520.

The color data acquisition process for each of the cloud point color computation at 503, the mesh vertex color computation at 505, or the texture computation at 506, is illustrated and described in more detail in FIGS. 6 and 7A-7C and their corresponding descriptions provided herein below.

FIG. 6 is a flow diagram illustrating a 3D scan process according to an embodiment. At 602, an image and metadata capture process is performed and an initial image set—and its corresponding metadata—is acquired. The image set includes at least a depth image and a color image. The depth image provides, for each pixel in a 2D image, a depth value while the color image provides, for each pixel in a 2D image, an RGB value. The image capture process by which the depth image and the color image are acquired is illustrated and described in more detail in FIGS. 7A-7C and the corresponding descriptions thereof, which are provided herein below. The image set acquired by the image capture process can be immediately stored, typically as a set of composite images, in a non-volatile memory and/or kept in volatile memory for some period of time.

At 603 through 607, a series of evaluations are performed in order to determine whether the color image captured at 602 will be elected as a candidate image for use in the computation of a color information value. The scanner continually acquires images and frames at a constant frame rate irrespective of whether scanner motion occurs between consecutive image/frame captures. However, newly acquired color images can be discarded—or previously acquired color images can be deleted, as appropriate—in order to limit the total amount of data that is written to memory (thereby decreasing the size of the scan file) while ensuring that high quality data is acquired, stored, and subsequently utilized in the construction of a point cloud, 3D mesh, and texture atlas.

At 603, the process evaluates whether a color image in the same neighborhood as the color image captured at 602 has previously been captured and saved, e.g., to non-volatile memory. In order to determine whether such a color image has been saved, previously saved color images are searched in order to determine whether the linear and/or angular position of the scanner during the image and metadata capture process of 602 are within a displacement threshold of the linear and angular positions of the scanner during the previous capture processes that provided the previously saved color images. An in-memory data structure can be utilized to store the metadata (captured in 602 and in previous image and metadata capture processes) in a manner that facilitates such a search. The color image captured at 602 is determined to be in the same neighborhood as a previously saved color image if (i) the scanner position during the capture of the color image at 602 is within a threshold distance of a scanner position during the capture of a respectively previously saved color image and (ii) the scanner orientation during the capture of the color image at 602 is within a threshold rotation of the scanner orientation during the capture of the respective previously saved color image. The displacement threshold therefore has two components: a translational component and a rotational component. The translational component is driven by the field of view of the cameras of the scanner: if the cameras have a broader field of view (FOV), the displacement threshold can be higher to avoid excessive data duplication in subsequent images. However, if the cameras have a narrower FOV, the threshold should be lower to ensure that the final set of saved texturing images have sufficient overlap to avoid voids in the final texture. The rotational component relates to the angular motion of the scanner. If the scanner is stationary but its orientation changes sufficiently, the new image may see part of the model that was occluded in a previously acquired image that was acquired from a different scanner orientation.

If the process determines, at 603, that there is no previously captured candidate color image in the neighborhood in which the scanner was located during the capture of the color image at 602, the process stores, at 604, the color image captured at 602 as a candidate color image, i.e. as an image that has been elected for consideration when coloring or texturing a 3D model. Storing the color image captured at 602 as a candidate color image can include, e.g., storing the candidate color image at a designated location in non-volatile memory and deleting the candidate color image from volatile memory. Thereafter, the process proceeds to 607 where it is determined whether the scan is complete.

Alternatively, if the process determines that there is a previously captured candidate color image (specifically, a previously saved color image) in the neighborhood in which the scanner was located during the capture of the color image at 602 (i.e. the “new color image”), the process evaluates, at 605, whether the new color image represents an improvement over the previously saved color image in the same neighborhood. In determining whether the new color image represents an improvement over the previously saved color image in the same neighborhood, the process evaluates whether the new color image was captured from a location closer to the target object, e.g. an oral structure, as compared to a capture position of the previously saved color image in the same neighborhood. The process can also evaluate, in determining whether the new color image represents an improvement, the speed of movement of the scanner during acquisition of the new color image and during acquisition of the previously saved color image in the same neighborhood.

If the process determines, at 605, that the new color image represents an improvement, the previously saved color image in the same neighborhood is deleted at 606. Thereafter, the process stores, at 604, the new color image (captured at 602) as a candidate color image, i.e. as an image that has been elected for consideration when coloring or texturing a 3D model. Alternatively, if the process determines that the new color image would not be an improvement, the process proceeds to 607 where it is determined whether the scan is complete.

At 607, the process evaluates whether the scan is complete. The scan is complete when a user explicitly terminates the scan process in order to stop the capture of image and metadata at 602. Accordingly, until the user terminates the scan process, the scanner continues to acquire image and metadata at 602 and process the captured data as described at 603-606 until user input terminating the scan is received at 607. When the scan is complete, the process stores metadata for each candidate color image at 608. The metadata can be stored as a single file and can include, for each image, a timestamp, a capture position, a capture orientation, a scanner movement speed, and camera calibration information. Thereafter, the process ends.

FIG. 7A is a flow diagram illustrating an image capture process according to an embodiment. At 701, the scanner, such as the intraoral scanner of FIG. 1, projects patterned ultraviolet (UV) light and captures an image while the patterned UV light is being projected. The captured image is an image of an object being scanned, e.g. an oral structure. The image captured at 701 is subsequently used to compute a depth image, which provides a depth value for each pixel thereof. The depth image is then, along with a number of other depth images, used to compute a 3D geometry of the object being scanned.

At 702, the scanner projects a uniform red light and captures an image (R image) while the uniform red light is projected thereon. At 703, the scanner projects a uniform green light and captures an image (G image) the uniform green light is projected thereon. At 704, the scanner projects a uniform blue light and captures an image (B image) while the uniform blue light is projected thereon. The images captured at 702, 703, and 704 are also images of the object being scanned, e.g. the oral structure. The images acquired at 701, 702, 703, and 704 collectively constitute a single frame of four images: a UV image, an R image, a G image, and a B image. In alternative aspects of the present disclosure that differ from that illustrated in FIG. 7A, a single color image can be acquired (e.g. using white light and a multi-chromatic image sensor) instead of the series of color images as illustrated in FIG. 7. In an embodiment, each of the UV, R, G and B images are acquired from monochromatic cameras that sense light in the UV, R, G and B range. The individual UV, R, G and B images captured by the respective UV, R, G and B monochromatic cameras, may then be shift and/or time-corrected relative to one another, and combined into a single composite image from which each of the UV, R, G, and B channel information can be extracted or set. When referring generically to an “image” herein, the term image may refer to a composite image that contains UV, R, G and B information in the same file, or may refer to individual monochromatic UV, R, G and B images.

The acquisition of the monochromatic images at 701, 702, 703, and 704 is performed at a constant rate such that the time difference between the capture of each successive monochromatic image in the frame is constant. For example, the images may be captured at a rate of 120 images per second, which corresponds to a period of slightly more than 8 milliseconds between the capture of consecutive images. Because the scanner is movable and may therefore move during the capture of consecutive images, a pixel from a first location in one image (e.g. the R image) may correspond to the same point of an object to be scanned that is located at a pixel in a different second location in a second image (e.g. the G image). In order to align the different images such that pixels in a same location in different monochromatic images of a frame correspond to the same point of the object to be scanned, it may be necessary to shift the images to compensate for said scanner movement.

At 705, the UV image captured at 701, the R image captured at 702, the G image captured at 703, and the B image captured at 704 are assembled into a single composite image. The composite image includes channels (each of which corresponds to a respective image captured at 701 through 704 and which can be accessed independently) and provides, for each pixel, a depth value and an RGB value. The UV and each of the RGB values are inherently shifted in relation to one another as a result of the time difference between individual channels. Furthermore, because the 3D point cloud is constructed from the depth data provided by the UV channel, the RGB values of the composite image are inherently shifted in relation to the points of the 3D geometry corresponding to the UV values of the composite image.

FIGS. 7B and 7C illustrate the impact of scanner movement on the capture position of consecutive monochromatic images of a single frame and the application of a channel shift correction to produce a corrected composite color image. As can be seen in FIG. 7B, movement of the scanner results in different capture positions for the different cameras: the UV image at time t, the R image at time t+x, the G image at time t+2x, and the B image at time t+3x. As can also be seen in FIG. 7C, combining the four images and performing a channel shift correction—whereby the R, G, and B images are shifted according to scanner movement to ensure that the pixels thereof are properly aligned with the pixels of the UV image—provides a corrected composite color image that provides an R, G, and B value for each pixel thereof.

FIG. 8 illustrates a process for coloring a 3D model. At 801A or 801B, the process loads the point cloud (801A) or the triangle mesh (801B) from memory, along with, at 801C, a set of composite scan images acquired during the scan of the object being modeled and stored to memory and, at 801D, image metadata associated with the composite scan images acquired during the scan. In some implementations of the process, each respective image file loaded from memory includes metadata associated with that image, while in alternative implementations, each respective image file includes an identifier that corresponds to a set of metadata stored within a larger metadata file. The image metadata associated with each respective composite scan image includes, e.g., an image capture position that identifies a point in a 3D coordinate system at which the scanner (or some component thereof, e.g. an individual camera in the scanner) is located, a scanner movement speed (i.e. a speed with which the intraoral scanner is moving during the capture of the respective image or the frame that includes the respective image), and an orientation (e.g. an angular position) of the intraoral scanner during the capture of a particular image. The image metadata is computed, by the intraoral scanner based on data provided by sensors located therein/thereon, during the acquisition of an image or of a set of images that constitute a frame.

At 802A, the process performs an occlusion test in order to determine, in the consideration of candidate images to potentially be used in determining the color of a respective point on the 3D mesh, the rapid elimination of composite scan images in which the respective point on the 3D mesh is occluded (due to another object in the 3D space that blocks the respective point from view. In the process illustrated in FIG. 8, the occlusion test is performed by computing an occlusion octree and eliminating from consideration those images in which the respective point is occluded. Octree occlusion culling is a well-known hierarchical occlusion culling technique that is frequently used in the computer graphics field. However, alternative techniques for determining whether the respective point on the 3D mesh is occluded (i.e. not visible) in individual composite images can also be utilized.

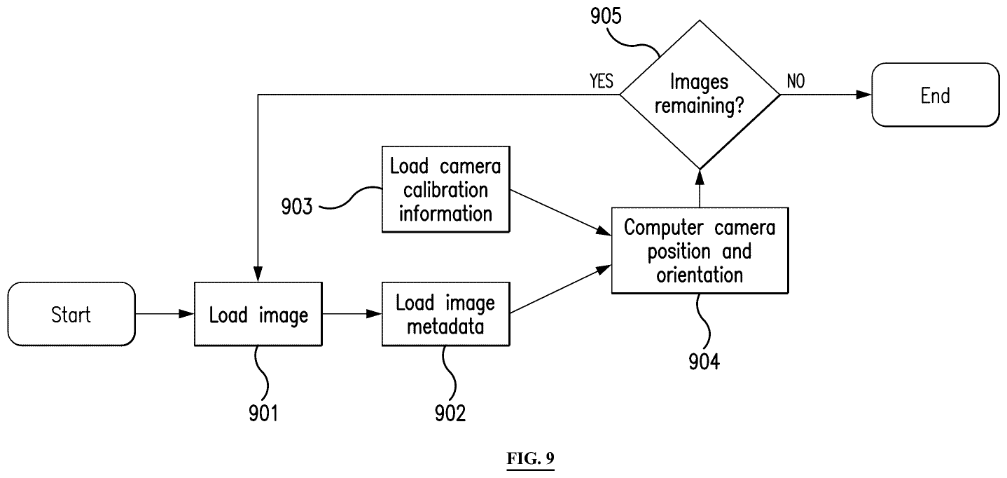

At 802B, the process creates frame objects. The frame objects are data structures that that allow for each of the composite images (e.g. as constructed at 705), and more specifically, the color data of each channel of said composite images (e.g. as acquired at 702 through 704), to be accurately projected onto the 3D mesh. An example of a process by which the frame objects can be created at 802B is illustrated and described in more detail in FIG. 9 and its corresponding description provided herein below.

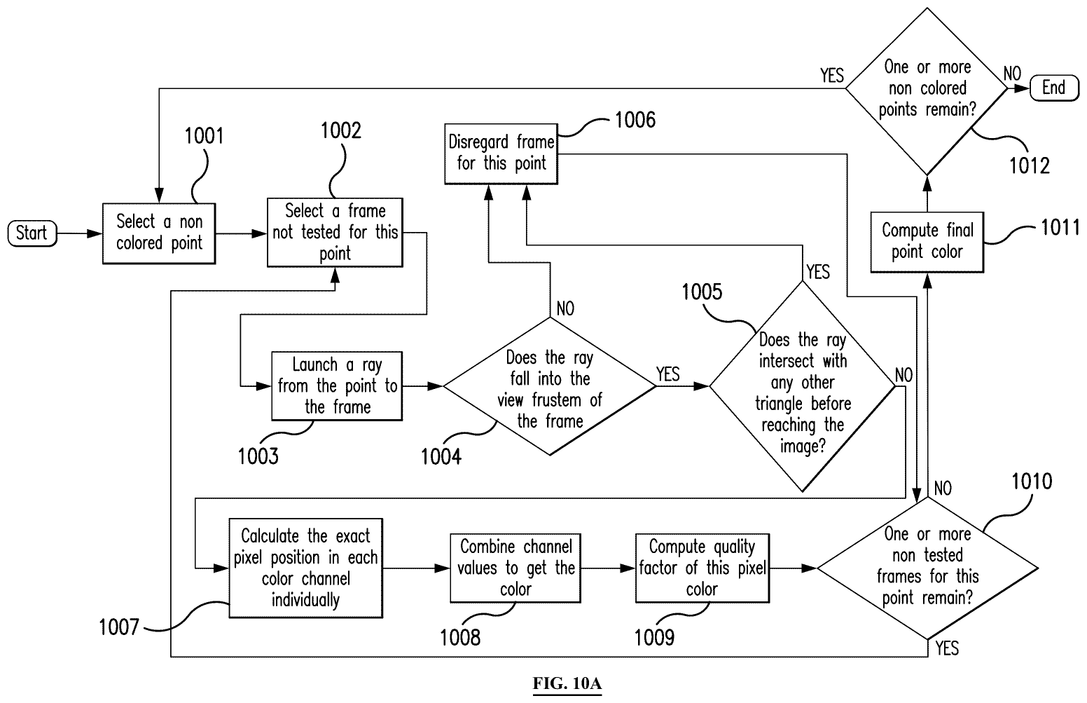

If the 3D model to be colored is a point cloud, at 803, the process computes the color for each point of the point cloud based on a set of candidate images from the collection of composite scan images that remain after occlusion culling at 802A. An example process for computing color of each point of a point cloud is illustrated and described in more detail in FIG. 10A and its corresponding description provided herein below.

If the 3D model to be colored is a 3D mesh based on a vertex coloring technique, at 804, the process may compute the color for each vertex of the 3D mesh based on a set of candidate images from the collection of composite scan images that remain after occlusion culling at 802A. An example process for computing color of each vertex of the 3D mesh is illustrated and described in more detail in FIG. 10A and its corresponding description provided herein below.

If the 3D model to be colored is a 3D mesh to be colored using a texturing technique, at 805, the process may compute a color for each point of each surface primitive of the 3D mesh. For example, for a 3D triangle mesh, the process computes, for each triangle that constitutes a part of the mesh, a color for each vertex, for two points on each edge (e.g. at 33% and 66% of the edge length), and for a single point in the center. An example process for computing color of each triangle of a 3D triangle mesh is illustrated and described in more detail in FIGS. 10A-10C and corresponding description provided herein below.

At 806, the process creates texture tiles based on the color computed at 805. For texturing a triangle mesh, each texture tile is a square 2D array of texels that contains the color information computed at 805 for a pair of triangles. Accordingly, the shared edge of the pair of triangles is represented by the diagonal of the texture tile, the four corners of the texel tile are vertex colors, the two texels on each side of the tile are edge colors, and the remaining two texels are centroid colors. An example of a single texture tile is depicted in FIG. 10B. At 807, the process creates a texture atlas file. The texture atlas file is an image file that includes all of the tiles for the entire 3D triangle mesh. The texture atlas is constructed by laying out all texture tiles in a grid-like fashion. FIG. 10C illustrates a texture atlas at different levels of resolution. At 808, the process creates a 2D-to-3D tile imagemapping that, for each vertex of the triangle mesh with 3D coordinates (x,y,z), gives the 2D coordinates (u,v) of its matching pixel in the texture map. Once the texture atlas file and 2D-to-3D mapping is complete, then in order to display/render the textured 3D model on a display, the 3D triangle mesh (downloaded at 801B), the texture atlas file (created at 807), and the 2D-to-3D mapping therebetween (created at 808) can be provided to a rendering engine which can render, for display, the textured 3D model.

FIG. 9 illustrates a process for creating frame objects. At 901, the process loads a composite scan image from memory (e.g. as constructed at 705), and at 902, the process loads metadata, associated with the respective composite scan image, from memory. The composite scan image is, e.g., stored as a JPG file, while the metadata is, e.g., stored as part of a binary file that includes metadata for a large number of composite scan images or as a smaller binary file that only includes the metadata for the respective composite scan image. The metadata can include, e.g., a timestamp, an image capture location (defined in a global 3D coordinate system maintained by the intraoral scanner during the scan), a scanner movement speed (i.e. a speed with which the intraoral scanner is moving during the capture of a particular image or frame consisting of multiple images), and an orientation of the intraoral scanner (e.g. an angle of rotation and degree of longitudinal and/or transverse tilt of the scanner during the capture of a particular image or frame consisting of multiple images). A collection of metadata can be matched to a particular composite scan image based on the timestamp (which can also be stored as part of the image file) or by an identifier that corresponds to both the relevant collection of metadata and the image file.

At 903, the process loads camera calibration information from memory. The camera calibration information provides a relationship between the position and orientation of the camera (or for an intraoral scanner that includes multiple cameras, the position and orientation of each of the multiple cameras) and the intraoral scanner's position and orientation. In other words, the camera calibration information allows the process to determine, based on a position and orientation of the intraoral scanner—as recorded by the intraoral scanner as metadata, an exact location and orientation of the camera (or cameras) that acquired the composite scan image (or the individual monochrome images that were combined at 705 to form the composite image) when the image(s) was(were) acquired.

At 904, the process computes the position and orientation of the camera for each of the color images that were captured during the scan. In particular, the process computes, using the image metadata and the camera calibration information as input, an exact location of the camera that acquired the monochrome data for each color channel of the composite scan image when said monochrome data was acquired. For example, the process determines, based on the scanner position and the scanner movement speed that correspond to a respective composite scan image, a scanner position that corresponds to each color channel of the respective composite scan image. Depending on the metadata that is recorded, the process can also determine, in some implementations, a scanner orientation that corresponds to each color channel of the composite scan image if a rate of change of the scanner orientation is also provided in the collection of metadata matched to the composite scan image. Alternatively, the scanner orientation stored in the collection of metadata can be assumed for each color channel of the composite scan image. Thereafter, the process can determine, using the camera calibration information and the scanner position and orientation for each respective color channel of a composite scan image, the exact location of the camera that acquired the monochrome image that provided the data for the respective color channel when the respective monochrome image was acquired. In this manner, the process provides compensation of the channel shift described, e.g., in FIGS. 7B and 7C and their corresponding description provided herein. Specifically, for each respective color channel of each composite scan image, the process provides a camera position and orientation coupled with respective color data. The camera position and orientation can be, e.g., a planar surface in the global 3D coordinate system that represents the surface of the camera's image sensor.

At 905, the process determines whether there are further composite scan images remaining for which frame objects (i.e. combinations of color channel data coupled with a camera position and orientation) have not yet been created. If additional images remain, the process returns to 901 where a new composite scan image is loaded. If no images remain, the process ends.

FIG. 10A illustrates a process for computing color for each point in a 3D model (e.g., in a 3D point cloud or a 3D mesh) or a texture to be applied to a 3D mesh (i.e. for computing a color for each texel of a texture atlas). At 1001, the process selects a non-colored point in the point cloud or the 3D mesh (as appropriate for the type of coloring being pursued—e.g., point cloud coloring, mesh vertex coloring, or texture coloring). In an embodiment, the implementation depicted in FIG. 10A may be used to calculate point colors of a texture generated for a 3D model that is a 3D triangle mesh, and each triangle includes 10 points that each correspond to a single texel in the texture atlas: 3 vertex points, 2 points on each of the three triangle edges, and a single point in the interior of the triangle (i.e. a centroid) (see FIG. 10B). In creating the texture atlas, triangles in the 3D triangle mesh are paired to form single 4×4 texel tiles. In this manner, the 2 shared vertex points and the 2 points of the shared edge of the triangle can each be represented, for both triangles, by a single texel. In other words, colors for each point of a triangle pair can be represented by 16 texels in the texture atlas.

At 1002, the process enters a loop whereby each frame object of the set of frame objects created at 802B is considered as a candidate for contributing color information to the point selected at 1001. Specifically, at 1002, the process selects a frame object not yet tested for its suitability for contributing color information to the point selected at 1001. At 1003, the process launches a ray from the point to the frame object. More specifically, the process launches a ray from the point selected at 1001 to a position of a camera during the acquisition of an image of the selected frame object. For the position of the camera during the acquisition of the color channel data of the selected frame object, the process can use the image capture location of a composite image corresponding to the selected frame object (which is stored as metadata associated with said composite image). Alternatively, the process can use a camera position of the frame object (e.g. a position and orientation of a camera as determined at 904 for the composite image) or a camera position of an individual color channel of the frame object (e.g. as determined at 904).

At 1004, the process determines, for the frame object selected at 1002 and the ray launched at 1003, whether the ray lies within the view frustum of the frame object. If the ray launched at 1003 does not lie within the view frustum of the frame object selected at 1002, the process proceeds to 1006 where the frame is disregarded for the point selected at 1001. To disregard the frame, the process can, for example, mark the frame with a temporary designation that is cleared when the process reaches 1010. After the frame is disregarded, the process proceeds to 1010—where it is determined whether additional, non-tested frame objects remain for the point selected at 1001. If, however, the ray launched at 1003 does lie within the view frustum of the frame object selected at 1002, the process proceeds to 1005.

At 1005, the process performs an occlusion test to determine whether the ray launched at 1003 intersects any other portion point in the point cloud, or any other portion of the triangle mesh, during its path from the point selected at 1001 to the position of the scanner. If the ray launched at 1003 does intersect a point cloud point or the triangle mesh on its path to the position of the scanner, then the point selected at 1001 is, in the selected frame object, obstructed from the view of the camera (i.e. it is obstructed by another point in the point cloud or another portion of the triangle mesh and does not appear in the images of the selected frame object). If the ray launched at 1003 is determined to intersect another point in the point cloud or another portion of the triangle mesh, the process proceeds to 1006 where the selected frame object is disregarded for the point selected at 1001. If, however, the ray launched at 1003 does not intersect the triangle mesh, the process proceeds to 1007.

At 1007, the process calculates the exact pixel position of the point selected at 1001 individually in each color channel of the frame object selected at 1002. Specifically, the process calculates, at 1007, the exact pixel of each respective color channel image of the composite image of the selected frame object. As described in connection with FIG. 9 above, the frame object provides, for each of multiple different color channels, a monochrome image and an associated camera position and orientation. At 1007, the process calculates, for each respective monochrome image and based on the associated camera position and orientation of said respective monochrome image, a pixel that corresponds to the point selected at 1001. The process thereby provides, at 1007, a monochrome pixel value—for each respective color channel—of the point selected at 1001. At 1008, the process combines the respective monochrome pixel values to provide, from the frame object selected at 1002, a corrected pixel color for the point selected at 1001.

At 1009, the process computes a quality factor of the corrected pixel color identified at 1008. In order to compute the quality factor of the corrected pixel color, the process can consider a number of different criteria. For example, the process can consider the distance, in the global 3D coordinate system, from the point selected at 1001 to the position of the camera, i.e. the position of the camera used in launching the ray at 1003, as well as the difference between said distance and the focal distance of the camera that acquired the color data of the frame object. The process can also consider the scanner movement speed (as stored in metadata) that corresponds to the frame object, as well as the degree of perpendicularity between the camera and the point selected at 1001. In order to determine the degree of perpendicularity between the camera and the point selected at 1001, the process can determine an angle between a normal to the point selected at 1001 and a view vector of the camera (which can be determined using the position and orientation of the camera, e.g. as determined for the frame object at 904). In a 3D mesh, in order to determine the normal to the point selected at 1001, the process takes into account the type of point. If the point selected at 1001 is a centroid, the normal extends in a direction perpendicular to a plane in which the triangle, to which the centroid corresponds, lies. If the point selected at 1001 is on a respective edge of a triangle (and more specifically, on a single respective edge shared by two triangles), the normal extends in a direction that is an average of a first direction perpendicular to a plane in which a first triangle, which shares the respective edge, lies and a second direction perpendicular to a second triangle, which also shares the respective edge, lies. If the point selected at 1001 is a vertex, the normal can be computed from the normals of all adjacent triangles. The computation of the normal for a vertex point can vary from one embodiment to another—or even vary from one point to another in a single embodiment. For example, the computation of the normal for a vertex point can be an average of the normals of all adjacent triangles. Alternatively, the computation of the normal for a vertex point can take into account the internal angle of the triangle at the vertex in order to determine a scaling factor for weighting the contribution of the normals of adjacent triangles.

In determining the quality factor of the corrected pixel color at 1009, the process can utilize different weighting factors. Different weighting factors can be chosen in order to consider different attributes of a frame to determine its quality. For example, low perpendicularity of the camera sensor plane with respect to the normal of the point of interest can indicate that the pixel is from an image in which the camera views the point of interest from too large of an angle. A high scanner movement speed can indicate a higher likelihood of blurriness. In addition, a high movement speed can greater channel shift in the composite color image—which can be harder to correct. A pixel that is too far from the center of the image can have a higher likelihood of being distorted if there is distortion on the edge of the image. A large distance of the point to the camera can indicate a higher likelihood that the color may have been impacted by light intensity falloff. A high degree of whiteness the pixel itself can indicate that it is a specular pixel, and the color has been unduly impacted by a reflection.

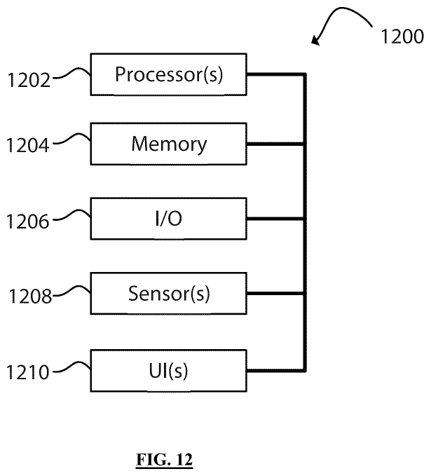

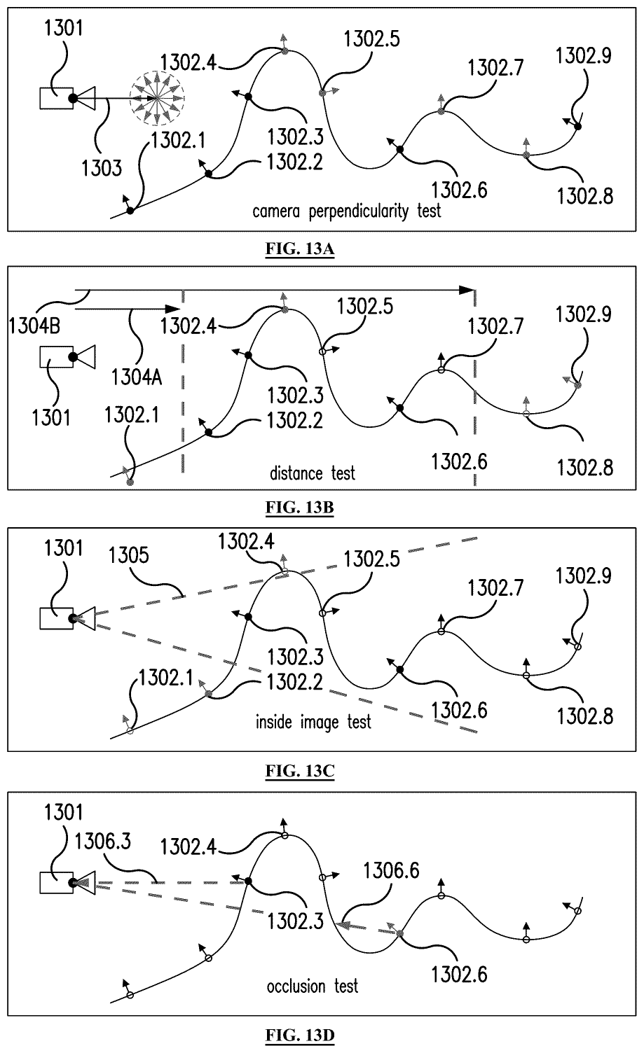

FIGS. 13A and 13B illustrate tests that can be performed in conjunction with the determination of the quality factor at 1009 of the corrected pixel color for the point selected at 1001. In addition to being used in conjunction with the determination of the quality factor at 1009, the tests illustrated in FIGS. 13A and 13B can also be used in the process illustrated in FIG. 10A, in combination with the view frustum analysis performed at 1004 and the occlusion test performed at 1005, to determine that the frame object selected at 1002 should be disregarded for the point selected at 1001. FIG. 13C illustrates a view frustum analysis for determining whether the point selected at 1001 is inside the image corresponding to the frame object selected at 1002 (e.g. the composite image used to create, via the process of FIG. 9, the frame object selected at 1002 or one or more individual monochrome images of such composite image). The view frustum analysis illustrated in FIG. 13C can be used, e.g., at 1004 of the process illustrated in FIG. 10A. FIG. 13D illustrates an occlusion test for determining whether the point selected at 1001 is, in the image corresponding to the frame object selected at 1002, obstructed from the camera by a portion of the triangle mesh. In order to determine whether the point selected at 1001 is obstructed from the camera in the image corresponding to the selected frame object, the occlusion test can determine whether a ray launched from the point selected at 1001 to a position of the camera intersects the triangle mesh. The occlusion test illustrated in FIG. 13D can be used, e.g., at 1005 of the process illustrated in FIG. 10A. In the case of a point cloud 3D model, the occlusion test can determine whether a ray launched from the point selected at 1001 to a position of the camera intersects another point along the ray between the camera and the point selected at 1001. If it does, the point selected at 1001 is occluded.

FIG. 13A illustrates a camera perpendicularity test in which a degree of perpendicularity between a normal to the point selected at 1001 (e.g. one of the points 1302.1, 1302.2, . . . , 1302.n) and the camera sensor plane of the camera 1301 is determined. In order to determine the degree of perpendicularity between the normal to the point of interest and the camera sensor plane, an angle between the normal to the point and a view vector 1303 of the camera (i.e. a normal to the camera sensor plane, which can be determined using the position and orientation of the camera, e.g. as determined for the frame object at 904) can be calculated. Typically, the greater the degree of perpendicularity between the normal to the point selected at 1001 and the camera sensor plane, the better the quality of the color data provided by the corresponding image and the higher the quality factor determined at 1009. In embodiments where the camera perpendicularity test is used to determine whether to disregard the frame object selected at 1002 for the point selected at 1001 (i.e. where the camera perpendicularity test is used in combination with 1004 and 1005 of the process illustrated in FIG. 10A), the selected frame object is disregarded for the point of interest if the degree of camera perpendicularity falls below a threshold. For example, if the angle between the normal to the point and the view vector 1303 of the camera falls outside a particular range, then the selected frame object is disregarded. For example, in FIG. 13A, frame objects with the camera orientation corresponding to the view vector 1303 would be disregarded for the points 1302.4, 1302.5, 1302.7, and 1302.8.

FIG. 13B illustrates a distance test in which a distance from the camera 1301 to the point selected at 1001 (e.g. on of the points 1302.1, 1302.2, . . . , 1302.n) is determined. The distance test can, for example, determine the distance from the camera 1301 to the point of interest relative to a focal length of the camera 1301. Typically, the closer the focal length of the camera to the distance from the camera to the point of interest, the better the quality of the color data provided by the corresponding image and the higher the quality factor determined at 1009. In embodiments where the distance test is used to determine whether to disregard the frame object selected at 1002 for the point selected at 1001 (i.e. where the distance test is used in combination with 1004 and 1005 of the process illustrated in FIG. 10A), the selected frame object is disregarded for the point of interest if the distance (for the frame object selected at 1002) from the camera to the point of interest is less than a lower threshold (i.e. 1304A in FIG. 13B) or greater than an upper threshold (i.e. 1304B in FIG. 13B). For example, in FIG. 13B, frame objects with the camera position corresponding to that of camera 1301 would be disregarded for the point 1302.1 due to the camera distance falling below the lower threshold 1304A and would also be disregarded for the points 1302.8 and 1302.9 due to the camera distance exceeding the upper threshold 1304B.

FIG. 13C illustrates an inside image test, i.e. a test to determine whether the point selected at 1001 lies within the view frustum of an image corresponding to the frame object selected at 1002. The inside image test can be performed, e.g., at 1004 of the process of FIG. 10A, and can be used to determine whether the point selected at 1001 is visible in the image (e.g. the composite image used to create, via the process of FIG. 9, the frame object selected at 1002 or one or more individual monochrome images of such composite image) corresponding to the frame object selected at 1002. For example, in FIG. 13C, frame objects corresponding to the view frustum 1305 would be disregarded for the points 1302.1, 1302.2, and 1302.4.

FIG. 13D illustrates an occlusion test, i.e. a test to determine whether the point selected at 1001 is obstructed from the view of the camera 1301 in an image corresponding to the frame object selected at 1002. The occlusion test can determine whether a ray launched from the point selected at 1001 to a position of the camera 1301 intersects a point cloud point (in the case where the 3D model is a point cloud) or the triangle mesh (in the case where the 3D model is a 3D mesh or textured mesh) and can be used, e.g., at 1005 of the process illustrated in FIG. 10A. FIG. 13D illustrates a ray 1306.3 launched from the point 1302.3 to the camera 1301, and a ray 1306.6 launched from the point 1302.6 to the camera 1301. In the illustration of FIG. 13D, the ray 1306.6 intersects the surface connecting the points 1302.1, 1302.2, . . . , 1302.n. Therefore, frame objects corresponding to the position of the camera 1301 would be disregarded for the point 1302.6. In FIG. 13D, rays are not launched for other points that have previously been excluded in FIGS. 13A through 13C. In embodiments of the process described in FIG. 10A, once a frame object is disregarded for a point of interest, additional tests need not be performed in order to determine if that point should be disregarded on additional grounds as well.

Returning to FIG. 10A, at 1010, the process determines whether one or more non-tested frame objects for the point selected at 1001 remain. If one or more such frame objects remain, the process returns to 1002 and then proceeds to compute another quality factor for another intersected pixel color or disregard another frame object before returning to 1010. In contrast, no non-tested frame object for the point selected at 1001 remain, the process proceeds to 1011 where a final color for the point selected at 1001 is computed.

In order to compute, at 1011, the final color for the point selected at 1001, the process identifies a set of the N best corrected pixels based on the quality factors determined at 1009. The final color for the point selected at 1001 is then computed as a weighted average of the N best corrected pixels.

At 1012, the process determines whether one or more non-colored points remain. If one or more non-colored points remain, the process returns to 1001 where a new non-colored point is selected and then proceeds to compute a final point color for that point before returning to 1012. If no non-colored points remain, the process ends.

FIG. 11A illustrates a textured, i.e. colored, 3D virtual model 1101 of a tooth 1102, while FIG. 11B illustrates a 3D triangle mesh 1103 that corresponds to the textured 3D virtual model of the tooth of FIG. 11A. The texture provided to the 3D virtual model of FIG. 11A, which is provided in the form of a triangle mesh, is generated by the process of FIG. 5 and its component subprocesses, as described above. The texture created in this manner provides dental laboratories with enhanced color details that are critical for the cosmetic finishing of dental restorations and prostheses, and the improved texture additionally enables both patients and practitioners to make more informed decisions regarding potential treatment plans by better visualizing existing dental pathologies. The resolution of the texture created according to the aforementioned processes is independent from the resolution of the geometric information embodied by the triangle mesh, and very fine color detail are provided even on surfaces constructed from very coarse geometric details (e.g. large triangles).

FIG. 12 is a block diagram of an exemplary processing system, which can be configured to perform operations disclosed herein. Referring to FIG. 12, a processing system 1200 can include one or more processors 1202, memory 1204, one or more input/output devices 1206, one or more sensors 1208, one or more user interfaces 1210, and one or more actuators 1212. Processing system 1200 can be representative of each computing system disclosed herein.

Processors 1202 can include one or more distinct processors, each having one or more cores. Each of the distinct processors can have the same or different structure. Processors 1202 can include one or more central processing units (CPUs), one or more graphics processing units (GPUs), circuitry (e.g., application specific integrated circuits (ASICs)), digital signal processors (DSPs), and the like. Processors 1202 can be mounted to a common substrate or to multiple different substrates.

Processors 1202 are configured to perform a certain function, method, or operation (e.g., are configured to provide for performance of a function, method, or operation) at least when one of the one or more of the distinct processors is capable of performing operations embodying the function, method, or operation. Processors 1202 can perform operations embodying the function, method, or operation by, for example, executing code (e.g., interpreting scripts) stored on memory 1204 and/or trafficking data through one or more ASICs. Processors 1202, and thus processing system 1200, can be configured to perform, automatically, any and all functions, methods, and operations disclosed herein. Therefore, processing system 1200 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 1200 can be configured to perform task “X”. Processing system 1200 is configured to perform a function, method, or operation at least when processors 1202 are configured to do the same.

Memory 1204 can include volatile memory, non-volatile memory, and any other medium capable of storing data. Each of the volatile memory, non-volatile memory, and any other type of memory can include multiple different memory devices, located at multiple distinct locations and each having a different structure. Memory 1204 can include remotely hosted (e.g., cloud) storage.

Examples of memory 1204 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 1204.

Input-output devices 1206 can include any component for trafficking data such as ports, antennas (i.e., transceivers), printed conductive paths, and the like. Input-output devices 1206 can enable wired communication via USB®, DisplayPort®, HDMI®, Ethernet, and the like. Input-output devices 1206 can enable electronic, optical, magnetic, and holographic, communication with suitable memory 1206. Input-output devices 1206 can enable wireless communication via WiFi®, Bluetooth®, cellular (e.g., LTE®, CDMA®, GSM®, WiMax®, NFC®), GPS, and the like. Input-output devices 1206 can include wired and/or wireless communication pathways.

User interface 1210 can include displays, physical buttons, speakers, microphones, keyboards, and the like. Actuators 1212 can enable processors 1202 to control mechanical forces.

Processing system 1200 can be distributed. For example, some components of processing system 1200 can reside in a remote hosted network service (e.g., a cloud computing environment) while other components of processing system 1200 can reside in a local computing system. Processing system 1200 can have a modular design where certain modules include a plurality of the features/functions shown in FIG. 12. For example, I/O modules can include volatile memory and one or more processors. As another example, individual processor modules can include read-only-memory and/or local caches.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A method for generating a texture for a three-dimensional (3D) model of an oral structure, the method comprising:
providing the 3D model of the oral structure, the 3D model of the oral structure being provided in the form of a polygon mesh that includes a number of connected polygons registered in a 3D coordinate system;
identifying a set of points located on the polygon mesh, each respective point in the set of points being defined by a coordinate value in the 3D coordinate system;
determining, for each respective point in the set of points, a respective texture value, wherein each respective texture value is determined by:
identifying a set of frames,
filtering the set of frames to identify a subset of frames,
determining a set of candidate texture values for the respective texture value, each candidate texture value corresponding to a respective frame in the subset of frames,
computing, for each respective candidate texture value in the set of candidate texture values, a quality factor, and
computing the respective texture value for the respective point by combining, based on their respective quality factors, candidate texture values selected from the set of candidate texture values; and
creating a texture atlas, the texture atlas being provided in the form of a two-dimensional (2D) texture image, the 2D texture image including a number of texels, and a mapping between each respective texel in the 2D texture image and a corresponding point located on the polygon mesh in the 3D coordinate system,
wherein each respective texel in the 2D texture image has a value equal to the respective texture value determined for the respective point in the set of points that corresponds to the respective texel.

2. The method according to claim 1, wherein the set of points located on the polygon mesh includes, for each respective polygon in the polygon mesh, at least one point.

3. The method according to claim 1, wherein the set of points located on the polygon mesh includes, for each respective polygon in the polygon mesh, at least one vertex point, at least one edge point, and at least one interior point.

4. The method according to claim 1, wherein each polygon in the polygon mesh is a triangle, and wherein the set of points located on the polygon mesh includes, for each respective triangle in the polygon mesh, three vertex points, at least three edge points, and at least one interior point.

5. The method according to claim 1, wherein each frame in the set of frames includes a depth image and a composite color image, and wherein the 3D mesh is a 3D mesh constructed using depth data from the respective depth images.

6. The method according to claim 1, wherein each respective frame in the subset of frames includes a composite color image, the composite color image including a plurality of color channels.

7. The method according to claim 6, wherein determining each respective candidate texture value that corresponds to a respective frame in the subset of frames comprises:
determining, for each respective color channel of the plurality of color channels of the composite color image of the respective frame, a color channel contribution, and
combining each respective color channel contribution to provide the respective candidate texture value.

8. The method according to claim 7, wherein the composite color image of each frame in the subset of frames is a combination of monochrome images, each monochrome image corresponding to a respective color channel of the plurality of color channels.

9. The method according to claim 8, wherein determining the color channel contribution for each respective color channel of the composite color image comprises:
determining, based on a camera position in the 3D coordinate system that corresponds to the monochrome image corresponding to the respective color channel and the coordinate value in the 3D coordinate system of the respective point for which the respective texture value is computed, a pixel in the monochrome image and providing a pixel value of the determined pixel as the color channel contribution for the respective color channel.

10. The method according to claim 9, wherein each respective monochrome image of each composite image is independently associated with a respective camera position in the 3D coordinate system.

11. The method according to claim 1, wherein filtering the set of frames to identify the subset of frames includes performing, for each respective frame in the set of frames, at least one of: a camera perpendicularity test that analyzes a degree of perpendicularity between a camera sensor plane corresponding to the respective frame and a normal of the respective point located on the polygon mesh, a camera distance test that analyzes a distance, in the 3D coordinate system, between a camera capture position corresponding to the respective frame and the respective point located on the polygon mesh, a view frustum test that determines whether the respective point located on the polygon mesh is located in a view frustum corresponding to the respective frame, or an occlusion test that analyzes whether the point located on the polygon mesh is, in an image corresponding to the respective frame, obstructed by other surfaces of the polygon mesh.

12. The method according to claim 1, wherein computing, for each respective candidate texture value in the set of candidate texture values, a quality factor includes assigning, for each respective frame in the set of subframes, weighting factors based on at least one of: a degree of perpendicularity between a camera sensor plane corresponding to the respective frame and a normal of the respective point located on the polygon mesh, a distance, in the 3D coordinate system, between a camera capture position corresponding to the respective frame and the respective point located on the polygon mesh, a scanner movement speed corresponding to the respective frame, or a degree of whiteness of the respective candidate texture value.

13. The method according to claim 1, wherein computing the respective texture value for the respective point by combining, based on their respective quality factors, candidate texture values selected from the set of candidate texture values comprises: selecting a subset of the candidate texture values based on their respective quality factors and averaging individual color channel values provided by each candidate texture value in the subset of candidate texture values.

14. A non-transitory computer readable medium having processor-executable instructions stored thereon, the processor-executable instructions configured to cause a processor to carry out a method for generating a texture for a three-dimensional (3D) model of an oral structure, the method comprising:
providing the 3D model of the oral structure, the 3D model of the oral structure being provided in the form of a polygon mesh that includes a number of connected polygons registered in a 3D coordinate system;
identifying a set of points located on the polygon mesh, each respective point in the set of points being defined by a coordinate value in the 3D coordinate system;
determining, for each respective point in the set of points, a respective texture value, wherein each respective texture value is determined by:
identifying a set of frames,
filtering the set of frames to identify a subset of frames,
determining a set of candidate texture values for the respective texture value, each candidate texture value corresponding to a respective frame in the subset of frames,
computing, for each respective candidate texture value in the set of candidate texture values, a quality factor, and
computing the respective texture value for the respective point by combining, based on their respective quality factors, candidate texture values selected from the set of candidate texture values; and
creating a texture atlas, the texture atlas being provided in the form of a two-dimensional (2D) texture image, the 2D texture image including a number of texels, and a mapping between each respective texel in the 2D texture image and a corresponding point located on the polygon mesh in the 3D coordinate system,
wherein each respective texel in the 2D texture image has a value equal to the respective texture value determined for the respective point in the set of points that corresponds to the respective texel.

15. A system for generating a texture for a three-dimensional (3D) model of an oral structure, the system comprising:
processing circuitry configured to:
provide the 3D model of the oral structure, the 3D model of the oral structure being provided in the form of a polygon mesh that includes a number of connected polygons registered in a 3D coordinate system;
identify a set of points located on the polygon mesh, each respective point in the set of points being defined by a coordinate value in the 3D coordinate system;
determine, for each respective point in the set of points, a respective texture value, wherein each respective texture value is determined by:
identifying a set of frames,
filtering the set of frames to identify a subset of frames,
determining a set of candidate texture values for the respective texture value, each candidate texture value corresponding to a respective frame in the subset of frames,
computing, for each respective candidate texture value in the set of candidate texture values, a quality factor, and
computing the respective texture value for the respective point by combining, based on their respective quality factors, candidate texture values selected from the set of candidate texture values; and
create a texture atlas, the texture atlas being provided in the form of a two-dimensional (2D) texture image, the 2D texture image including a number of texels, and a mapping between each respective texel in the 2D texture image and a corresponding point located on the polygon mesh in the 3D coordinate system,
wherein each respective texel in the 2D texture image has a value equal to the respective texture value determined for the respective point in the set of points that corresponds to the respective texel.

16. A method for coloring points in a three-dimensional (3D) model of an oral structure, the method comprising:
providing the 3D model of the oral structure, the 3D model of the oral structure comprising a plurality of points registered in a 3D coordinate system;
identifying a set of points in the plurality of points of the 3D model, each respective identified point in the 3D model being defined by a coordinate value in the 3D coordinate system;
determining, for each identified point in the 3D model, a respective color information value, the respective color information value determined by:
identifying a set of images captured from an image scan of at least a portion of the oral structure, the identified set of images each comprising a corresponding point that corresponds to the respective point in the 3D model and each having associated color information;
combining the color information associated with the corresponding point in each of the identified scan images into a color information value; and
associating the combined color information value with the respective color information value of the respective point in the 3D model.

17. The method according to claim 16, the 3D model of the oral structure comprising a point cloud comprising a plurality of points registered in a 3D coordinate system and representing the oral structure.

18. The method according to claim 16, the 3D model of the oral structure comprising a polygon mesh comprising a number of connected polygons registered in a 3D coordinate system, wherein the identified set of points are located on the polygon mesh.

19. The method according to claim 18, wherein the identified set of points comprise the vertices of the polygons in the polygon mesh.
